(19) United States
(12) Patent Application Publication
(10) Pub. No.: US 2014/0227213 A1
(43) Pub. Date: Aug. 14, 2014

(54) COSMETIC COMPOSITIONS FOR DEEPER ETHNIC SKIN TONES

(71) Applicant: L'OREAL, Paris (FR)

(72) Inventors: Shauna-Kaye Lecaine Dominique SCOTLAND, Parsippany, NJ (US); Gisela PERRUNA, Rahway, NJ (US); Ashini AMIN, Monroe, NJ (US); Janine CHERETTE, Middlesex, NJ (US)

(73) Assignee: L'OREAL, Paris (FR)

(21) Appl. No.: 13/766,931

(22) Filed: Feb. 14, 2013

(51) Int. Cl. A61K 8/891; A61Q 1/02; A61Q 19/00

(52) U.S. Cl. CPC A61K 8/891; A61Q 19/00; A61Q 1/02
USPC 424/63; 424/78.03

(57) ABSTRACT

Disclosed herein are cosmetic compositions comprising, in a cosmetically acceptable medium, (1) at least one silicone elastomer blend, (2) at least one film-forming polymer chosen from polyalkylsilsesquioxane resins, (3) at least one siloxane resin, (4) at least one polymeric filler, and (5) at least one mineral filler. Also disclosed herein are methods for making up and/or enhancing the appearance of a keratinous substrate, such as the skin, comprising applying said compositions to the keratinous substrate.

### FIELD OF THE DISCLOSURE

The disclosure relates to cosmetic compositions comprising, in a cosmetically acceptable medium, (1) at least one silicone elastomer blend, (2) at least one film-forming polymer chosen from polyalkylsilsesquioxane resins, (3) at least one siloxane resin, (4) at least one polymeric filler, and (5) at least one mineral filler. Cosmetic compositions according to various embodiments of the disclosure may have one or more improved properties, such as improved oil absorption, shine reduction, matte, texture, long-wear properties, and/or ability to impart true color, while also reducing dullness, whiteness, and/or ashiness of the skin. The disclosure further relates to methods for making up and/or enhancing the appearance of a keratinous substrate, such as the skin, comprising applying said compositions to the keratinous substrate.

### BACKGROUND

Foundations are known and used in the cosmetic field to impart color to the skin. It is also known that foundations can help to control oil and shine on the skin. However, when preparing foundations for deeper ethnic skin tones, and especially in the case of oil-absorbing and/or matte compositions, it has been found that such compositions tend to impart an undesirable, dull or ashy hue to the skin

Polymeric and mineral fillers are well-known in the cosmetic field. Such fillers are generally incorporated into cosmetic compositions as oil-absorptive components which tend to have good adherence to the skin and contribute to the matte properties of the compositions. However, the use of fillers in compositions can tend to give the user an undesirable ashy and/or dull appearance and may, in some cases, result in a dry skin feel. The amount of fillers necessary to adequately absorb oil and prevent shine generally also tends to whiten the formulation and give the ashy dull hue that becomes more apparent after the composition dries on the skin. This problem is greatly exacerbated in the case of foundations for women with deeper ethnic skin tones.

Thus, there is a need in the cosmetic industry to provide consumers having deeper ethnic skin tones with cosmetic products having improved properties such as improved oil absorption, shine reduction, matte, texture, long-wear properties, and/or ability to impart true color, while also reducing dullness, whiteness, and/or ashiness of the skin. As such, there is a continuous need to invent novel cosmetic compositions which demonstrate one or more of the above-mentioned improved properties.

It has now been surprisingly discovered that by incorporating (1) at least one silicone elastomer blend, (2) at least one film-forming polymer chosen from polyalkylsilsesquioxane resins, (3) at least one siloxane resin, (4) at least one polymeric filler, and (5) at least one mineral filler into cosmetic compositions, undesirable cosmetic properties such as ashiness, dullness, and/or whiteness may be reduced and, in some cases, eliminated.

### DESCRIPTION OF EXEMPLARY EMBODIMENTS

The disclosure relates, in various embodiments, to cosmetic compositions comprising, in a cosmetically acceptable medium, (1) at least one silicone elastomer blend, (2) at least one polyalkylsilsesquioxane resin, (3) at least one siloxane resin, (4) at least one polymeric filler, and (5) at least one mineral filler.

The cosmetic compositions may be useful, in certain embodiments, for users having deeper ethnic skin tones. The cosmetic compositions may, in some embodiments, impart true color while reducing the appearance of ashiness, whiteness, and/or dullness of the skin.

As used herein, the term “deeper ethnic skin tones” is intended to refer to those skin tones that are darker and tend to have more pronounced and deeper red, red/yellow, and yellow undertones. Users with deeper ethnic skin tones may belong to ethnicities including, but not limited to, African, African-American, Caribbean, Hispanic, Middle Eastern, and Indian ethnicities.

As used herein, the term “true color” is intended to refer to a color or shade that is most accurate or closest to the tone of the keratinous substrate to which it is applied. For instance, in at least certain exemplary embodiments, a composition that imparts true color according to the disclosure closely matches the skin tone without leaving an ashy, dull, white, and/or gray appearance. True color refers to the color obtained not only upon application to the keratinous substrate, but also after drying.

As used herein, the terms “ashy,” “ashiness” and variations thereof are intended to refer to the appearance of a gray overcast on the skin or keratinous substrate after the product has been applied and evaluated after drying.

Silicone Elastomer Blends

The cosmetic compositions disclosed herein comprise at least one silicone elastomer blend. Silicone elastomer blends useful according to various embodiments of the disclosure may comprise at least one silicone cross-polymer dispersed in at least one silicone oil.

The at least one silicone cross-polymer may, in certain embodiments, be chosen from dimethicone cross-polymers. Non-limiting examples of suitable dimethicone cross-polymers include those obtained by reacting 1,5-hexadiene with the reaction product of polydimethyl siloxane and dimethyl methyl hydrogen siloxane. In certain embodiments, the reaction product may be obtained by reacting about 70-90% of polydimethyl siloxane with about 10-90% dimethyl methyl hydrogen siloxane.

The silicone cross-polymers may be dispersed in at least one silicone oil. In certain embodiments, the oil may be chosen from cyclic and linear organopolysiloxanes. Cyclic organopolysiloxanes may include, for example, cyclotetrasiloxane; cyclopentasiloxane; and methylated cyclic organopolysiloxanes, e.g., octamethylcyclotetrasiloxane and decamethylcyclopentasiloxane. Non-limiting examples of linear organopolysiloxanes include low molecular weight dimethicones; high molecular weight dimethicones; alkyl derivatives of linear organopolysiloxanes, e.g., cetyl dimethicone and lauryl trimethicone; aryl derivatives of linear organopolysiloxanes, e.g., phenyl trimethicone; and hydroxylated derivatives of linear organopolysiloxanes, e.g., dimethiconol.

In at least one embodiment, the silicone oil may be a low viscosity oil, for example, an oil having a viscosity ranging from about 5 cSt to about 20 cSt, at approximately room temperature and pressure. In other embodiments, the silicone oil may be chosen from low viscosity dimethicones having a viscosity of about 5 cSt at approximately room temperature and pressure, and low viscosity dimethicones having a viscosity of about 20 cSt at approximately room temperature and pressure.

Non-limiting examples of commercially available silicone elastomer blends include the products sold by Dow Corning under the names Dow Corning® 9040 and Dow Corning® 9041, and Dow Corning® 9045.

The at least one silicone elastomer blend may, in various embodiments, be present in the cosmetic composition in an amount ranging from about 5% to about 15% by weight, relative to the total weight of the composition. By way of non-limiting example, the at least one silicone elastomer blend may be present in the cosmetic composition in an amount ranging from about 6% to about 14% by weight, such as from about 7% to about 13% by weight, from about 8% to about 12% by weight, or from 10% to about 13% by weight, relative to the total weight of the cosmetic composition, including all ranges and subranges therebetween.

Film-Forming Polymers

As used herein, the terms “film-forming polymer,” “film former” and variations thereof mean a polymer capable of, by itself or in the presence of an auxiliary film-forming agent, forming a continuous or substantially continuous film that adheres to a support, and especially to keratin materials, for instance, the skin.

The cosmetic compositions disclosed herein comprise at least one polyalkylsilsesquioxane film-forming resin. In some embodiments, the compositions may comprise one or more additional film-forming polymers, for example, vinyl (co)polymers, (meth)acrylic (co)polymers, and mixtures thereof. According to various embodiments, the at least one film-forming polymer is a polypropylsilsesquioxane resin. In other embodiments, the at least one film-forming polymer is a mixture of at least one polypropylsilsesquioxane resin and at least one acrylic film-forming polymer.

Silsesquioxane resins are a specific form of silicone resins. Silicone resin nomenclature is known in the art as “MDTQ” nomenclature, whereby a silicone resin is described according to the various monomeric siloxane units which make up the polymer. Each letter of “MDTQ” denotes a different type of unit.

The letter M denotes the monofunctional unit R₃SiO_{1/2}. This unit is considered to be monofunctional because the silicone atom only shares one oxygen when the unit is part of a polymer. The symbol D denotes the difunctional unit RSiO_{2/2} wherein two oxygen atoms bonded to the silicone atom are used for binding to the rest of the polymer. The symbol T denotes the trifunctional unit RSiO_{3/2}, wherein three oxygen atoms bonded to the silicone atom are bonded to the rest of the polymer. The symbol Q denotes the tetrafunctional unit SiC_{4/2}, wherein all four oxygens bonded to the silicone atom are bonded to the rest of the polymer. Non-limiting examples of suitable R groups in the M, D, and T units include alkyl groups, alkene groups, alkyne groups, hydroxyl groups, thiol groups, ester groups, acid groups, ether groups, phenyl groups, alkoxy groups, which may be optionally substituted.

When the film forming resin is made up predominantly of trifunctional units or T units, it is generally called a silsesquioxane resin. If R in the T unit is chosen, for example, from methyl, the resin is called a polymethylsilsesquioxane. A non-limiting example of the at least one polymethylsilsesquioxane film former is Belsil PMS MK, also referred to as Resin MK, available from Wacker Chemie AG. This polymethylsilsesquioxane film former is a polymer comprising polymerized repeating units of CH₃SiO_{3/2} (T units) and may also contain up to 1% by weight or by mole of units of the formula (CH₃)₂SiO_{2/2} (D units). The weight-average molecular weight of this polymer has been estimated to be 10,000.

When the film forming resin is made up predominantly of trifunctional RSiO_{3/2} T units, wherein R is a propyl radical, it is called a polypropylsilsesquioxane. Polypropylsilsesquioxane resins may be obtained, for example, via hydrolysis and condensation of propyltrichlorosilane. The resin may, in certain embodiments, be a mixture of a T-propyl resin or a propyl-T resin with a carrier solvent. The carrier solvent may be chosen from any suitable volatile solvent such as, for example, C₁₃-C₁₄ isoparaffins, isododecane, isoeicosane, and isohexadecane. In at least one embodiment, the volatile solvent is isododecane.

One example of a polypropylsilsesquioxane resin suitable for use in exemplary compositions is commercially available from Dow-Corning as Dow Corning® 670 Fluid. Dow Corning® 670 Fluid comprises approximately 50 wt % polypropylsilsesquioxane and approximately 50 wt % cyclopentasiloxane. Another example of a suitable polypropylsilsesquioxane resin is commercially available from Dow Corning as Dow Corning® 680 ID Fluid. Dow Corning® 680 ID Fluid comprises approximately 72 wt % polypropylsilsesquioxane and approximately 28 wt % isododecane.

The polypropylsilsesquioxanes in these commercial products have a general formula of RₙSiO_{(4-n)/2} wherein R is independently chosen from a hydrogen atom and a monovalent hydrocarbon group comprising 3 carbon atoms, wherein more than 80 mole % of R are propyl groups, n is a value ranging from 1.0 to 1.4, more than 60 mole % of the copolymer comprises RSiO_{3/2} units, and having a hydroxyl or alkoxy content ranging from about 0.2% to about 10% by weight, for example from about 1% to about 4% by weight, or from about 5% to about 10% by weight, such as from about 6% to about 8% by weight.

The at least one film-forming polyalkylsilsesquioxane may be present in the cosmetic compositions according to the disclosure in an amount ranging from about 2% to about 10% by weight, relative to the total weight of the cosmetic composition. In certain embodiments, the at least one film-forming polyalkylsilsesquioxane may be present in the cosmetic compositions in an amount ranging from about 2% to about 8% by weight, such as from about 3% to about 7% by weight, or from about 4% to about 5% by weight, relative to the total weight of the cosmetic composition, including all ranges and subranges therebetween.

When the cosmetic compositions disclosed herein comprise at least one polyalkylsilsesquioxane film-forming resin and at least one additional film-forming polymer, the additional film-forming polymer may be chosen, for example, from vinyl (co)polymers, such as acrylic polymers. The vinyl film-forming polymers can result from the polymerization of monomers comprising at least one ethylenic unsaturation and at least one acidic group and/or esters of these acidic monomers and/or amides of these acidic monomers.

Monomers comprising at least one acidic group which may be used include, for example, α,β-ethylenic unsaturated carboxylic acids such as acrylic acid, methacrylic acid, crotonic acid, maleic acid or itaconic acid. In at least one embodiment, the monomers are chosen from (meth)acrylic acid and crotonic acid.

The esters of acidic monomers may be chosen, for example, from (meth)acrylic acid esters (also known as (meth)acrylates), such as (meth)acrylates of an alkyl, for example, a C₁-C₃₀ alkyl, such as a C₁-C₂₀ alkyl, (meth)acrylates of an aryl, such as a C₆-C₁₀ aryl, and (meth)acrylates of a hydroxyalkyl, such as a C₂-C₆ hydroxyalkyl.

Among the alkyl (meth)acrylates that may be mentioned, examples include methyl methacrylate, ethyl methacrylate, butyl methacrylate, isobutyl methacrylate, 2-ethylhexyl methacrylate, lauryl methacrylate and cyclohexyl methacrylate. The hydroxyalkyl (meth)acrylates may include, but are not limited to, hydroxyethyl acrylate, 2-hydroxypropyl acrylate, hydroxyethyl methacrylate and 2-hydroxypropyl methacrylate. By way of non-limiting example, the aryl (meth)acrylates may be chosen from benzyl acrylate and phenyl acrylate. The (meth)acrylic acid esters that may be used include, for example, alkyl (meth)acrylates. As disclosed herein, the alkyl group of the esters may be either fluorinated or perfluorinated, i.e., some or all of the hydrogen atoms of the alkyl group are substituted with fluorine atoms.

Examples of amides of the acid monomers include, but are not limited to, (meth)acrylamides, such as N-alkyl(meth)acrylamides, for example, of a C₂-C₁₂ alkyl. Among the N-alkyl(meth)acrylamides that may be mentioned, examples include N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or esters thereof and/or amides thereof, such as those mentioned above. Examples of vinyl esters that may be mentioned include vinyl acetate, vinyl neodecanoate, vinyl pivalate, vinyl benzoate and vinyl t-butylbenzoate. Styrene monomers that may be mentioned include, but are not limited to, styrene and α-methylstyrene.

In various embodiments, the film-forming polymer may have a Tg (glass transition temperature) value of less than about 50° C. In other embodiments, the cosmetic compositions of the instant disclosure comprise at least one film-forming polymer in the form of particles dispersed in an aqueous phase, which is generally known in the cosmetic art as a latex.

Commercially available aqueous dispersions of film-forming polymers which may be used include, but are not limited to, the acrylic dispersions sold under the names “Neocryl XK-90®”, “Neocryl A-1070®)”, “Neocryl A-1090®)”, “Neocryl BT-62®”, “Neocryl A-1079®)” and “Neocryl A-523®” by the company Avecia-Neoresins, “Dow Latex 432®” by the company Dow Chemical, “Daitosol 5000 AD®)” or “Daitosol 5000 SJ” by the company Daito Kasey Kogyo; “Syntran 5760” or “Syntran 5760 CG” by the company Interpolymer; vinyl dispersions, for instance “Mexomer PAM” and also acrylic dispersions in isododecane, for instance “Mexomer PAP” by the company Chimex.

The at least one additional film-forming polymer may, in various exemplary embodiments, be present in the cosmetic compositions in an amount ranging from about 0% to about 3% by weight, relative to the total weight of the cosmetic composition. By way of non-limiting example, the at least one additional film-forming polymer may be present in the cosmetic compositions in an amount ranging from about 0% to about 2% by weight, such as from about 0.01% to about 1% by weight, or from about 0.1% to about 0.3% by weight, relative to the total weight of the cosmetic composition, including all ranges and subranges therebetween.

Siloxane Resins

The cosmetic compositions disclosed herein further comprise at least one siloxane resin. Suitable siloxane resins in accordance with the disclosure include “MQ” resins, which comprise at least 80 mole % of (R₃SiO_{1/2})ₐ units (M units) and (SiC_{4/2})_{b} units (Q units). In some embodiments, R can be chosen from C₁-C₈ alkyl groups, aryl groups, carbinol groups, and amino groups. According to other embodiments, at least 95 mole % of the R groups are chosen from alkyl groups. In still further embodiments, a and b are values greater than 0 and are chosen such that the ratio a/b ranges from about 0.5 to about 1.5.

The alkyl groups may, for example, be chosen from methyl, ethyl, propyl, butyl, pentyl, hexyl, and octyl groups. In certain embodiments, the alkyl group is chosen from methyl and propyl groups. The aryl groups may be chosen, for instance, from phenyl, naphthyl, benzyl, tolyl, xylyl, xenyl, methylphenyl, 2-phenylethyl, 2-phenyl-2-methylethyl, chlorophenyl, bromophenyl and fluorophenyl groups. According to various embodiments, the aryl group is a phenyl group.

As used herein, the term “carbinol group” is intended to mean any group containing at least one hydroxyl radical bonded to a carbon (COH). The carbinol groups may thus contain more than one COH radical, such as, for example, two or three or more COH radicals. In certain embodiments, the carbinol group is free of aryl groups and contains at least 3 carbon atoms. In other embodiments, the carbinol group comprises at least one aryl group and contains at least 6 carbon atoms.

Examples of carbinol groups free of aryl groups and containing at least 3 carbon atoms include, but are not limited to, groups of formula R¹OH in which R¹ represents a divalent hydrocarbon-based radical containing at least 3 carbon atoms or a divalent hydrocarbonoxy radical containing at least 3 carbon atoms. As examples of an R¹ group, mention may be made of alkylene radicals, such as -(CH₂)ₓ—, wherein x ranges from 3 to 10, —CH₂CH(CH₃)—, —CH₂CH(CH₃)CH₂—, —CH₂CH₂CH(CH₂CH₃)CH₂CH₂CH₂—, and —OCH(CH₃)(CH₂)ₓ—, wherein x ranges from 1 to 10.

Non-limiting examples of carbinol groups comprising aryl groups and having at least 6 carbon atoms include groups of formula R²OH in which R² represents an arylene radical, such as —(CH₂)ₓC₆H₄—, where x ranges from 0 to 10, —CH₂CH(CH₃)(CH₂)ₓC₆H₄—, where x ranges from 0 to 10, and —(CH₂)ₓC₆H₄(CH₂)ₓ—, where x ranges from 1 to 10. The carbinol groups comprising aryl groups may, in certain embodiments, generally comprise from 6 to 14 atoms.

As used herein, the term “amino group” is intended to denote groups of formula —R³NH₂ or —R³NHR⁴NH₂, R³ representing a divalent hydrocarbon-based radical containing at least two carbon atoms and R⁴ representing a divalent hydrocarbon-based radical containing at least 2 carbon atoms. The R³ group may, in various embodiments, represent an alkylene radical containing from 2 to 20 carbon atoms. As examples of an R³ group, mention may be made of ethylene, propylene, —CH₂CHCH₃—, butylene, —CH₂CH(CH₃)CH₂—, pentamethylene, hexamethylene, 3-ethylhexamethylene, octamethylene, and decamethylene groups. The R⁴ group may, for instance, be chosen from alkylene radicals containing from 2 to 20 carbon atoms. As examples of an R⁴ group, mention may be made of ethylene, propylene, —CH₂CHCH₃—, butylene, —CH₂CH(CH₃)CH₂—, pentamethylene, hexamethylene, 3-ethylhexamethylene, octamethylene, and decamethylene groups.

According to various embodiments, the amino groups are chosen from —CH₂CH₂CH₂NH₂ and —CH₂(CH₃)CHCH₂(H)NCH₃, —CH₂CH₂NHCH₂CH₂NH₂, —CH₂CH₂NH₂, —CH₂CH₂NHCH₃, —CH₂CH₂CH₂CH₂NH₂, —(CH₂CH₂NH)₃H, and —CH₂CH₂NHCH₂CH₂NHC₄H₉.

According to certain embodiments, the MQ resins may comprise D and T units, provided that at least 80 mol %, or at least 90 mol %, of the total siloxane units are M and Q units. The MQ siloxane resins can also contain residual hydroxyl groups, such as residual silanol (—SiOH) groups. In this case, the amount of —OH groups may range, for example, from about 2% to about 10% by weight of the MQ resin, such as from about 2% to about 5% by weight of the MQ resin.

Suitable MQ siloxane resins, as well as the methods for production thereof, are disclosed, for example, in U.S. Pat. Nos. 2,814,601 and 2,857,356, incorporated herein by reference in their entireties. Commercially available siloxane resins which may be used include, but are not limited to, the vinyl dimethicone/methicone silsesquioxane cross-polymer products sold by Shin-Etsu under the KSP product line, for example, KSP-100, KSP-101, and KSP-105.

The at least one siloxane resin may, in various exemplary embodiments, be present in the compositions in an amount ranging from about 0.5% to about 3% by weight, relative to the total weight of the cosmetic composition. For instance, the at least one siloxane resin may be present in an amount ranging from about 1% to about 3% by weight, or from about 1% to about 2% by weight, or from about 1.5% to about 2.5% by weight, relative to the total weight of the cosmetic composition, including all ranges and subranges therebetween.

Polymeric Fillers

The cosmetic compositions disclosed herein further comprise at least one polymeric filler. Polymeric fillers can include, for example, lamellar or nonlamellar, colorless or white polymeric particles. In certain embodiments, the polymeric filler may be chosen from polyamide powders, such as Nylon® or Orgasol® powders from Arkema; acrylic polymer powders, such as polymethyl methacrylate powders, polymethyl methacrylate/ethylene glycol dimethacrylate powders, polyallyl methacrylate/ethylene glycol dimethacrylate powders, and ethylene glycol dimethacrylate/lauryl methacrylate copolymer powders; cellulose poly-β-alanine and polyethylene powders; tetrafluoroethylene polymer powders, such as Teflon® powders; lauroyllysine; starch; polymeric hollow microspheres such as those of polyvinylidene chloride/acrylonitrile, for instance Expancel® from Nobel Industrie; acrylic acid copolymers, such as Polytrap® from Dow Corning; silicone resin microbeads, such as Tospearls® from Toshiba; elastomeric polyorganosiloxane particles, such as those obtained by polymerization of organopolysiloxane having at least two hydrogen atoms each bonded to a silicon atom and of an organopolysiloxane comprising at least two ethylenically unsaturated groups, for instance, two vinyl groups, in the presence of a platinum catalyst; and metal soaps derived from organic carboxylic acids comprising from 8 to 22 carbon atoms, such as from 12 to 18 carbon atoms, for example, zinc stearate, magnesium stearate, lithium stearate, zinc laurate, and magnesium myristate.

Non-limiting examples of suitable commercial acrylic polymer powder products include the polymethyl methacrylate powders sold under the name Covabead® LH85 by Wacker; the polymethyl methacrylate/ethylene glycol dimethacrylate powders sold under the names Dow Corning 5640 Microsponge® Skin Oil Adsorber by Dow Corning and Ganzpearl® GMP-0820 by Ganz Chemical; the polyallyl methacrylate/ethylene glycol dimethacrylate powders sold under the name Poly-Pore® L200 and Poly-Pore® E200 by Amcol; and the ethylene glycol dimethacrylate/lauryl methacrylate copolymer powders sold under the name Polytrap® 6603 by Dow Corning. Examples of commercially available silicone elastomer powders include, but are not limited to, the powders sold under the names Trefil® Powder E-505C and Trefil® Powder E-506C by Dow Corning.

In at least one exemplary embodiment, the at least one polymeric filler is a polyamide powder. According to further embodiments, the at least one polymer filler is chosen from Nylon-12, Nylon-6, and combinations thereof.

The at least one polymeric filler may, in various exemplary embodiments, be present in the cosmetic compositions in an amount ranging from about 0.5% to about 5% by weight, for example, from about 1% to about 3%, or from about 1.5% to about 2.5%, relative to the total weight of the composition, including all ranges and subranges therebetween. In various embodiments, the at least one polymeric filler is Nylon-12 and is present in an amount ranging from about 1% to about 5% by weight, such as about 2% by weight relative to the total weight of the cosmetic composition.

Mineral Fillers

The cosmetic compositions disclosed herein further comprise at least one mineral filler. Mineral fillers can include, for example, lamellar or nonlamellar, colorless or white mineral particles of any shape, such as spherical, platelet, or oblong particles. In certain embodiments, the mineral fillers may be chosen from talc, mica, silica, kaolin, hectorite, boron nitride, precipitated calcium carbonate, magnesium carbonate, magnesium hydrogen carbonate, hydroxyapatite, hollow silica microspheres, such as Silica Beads® from Maprecos, glass or ceramic microcapsules, clay, quartz, natural diamond powder, and mixtures thereof.

Non-limiting examples of commercially available silica powders include the porous silica microspheres sold under the name Silica Beads SB-700 by Myoshi Kasei, and Sunsphere® H51 and H33 by Asahi Glass; and the polydimethylsiloxane-coated amorphous silica microspheres sold under the names SA Sunsphere® H 33 and H53 by Asahi Glass.

According to various embodiments, the at least one mineral filler is chosen from silica powders, such as Sunsphere® H51 by AGC SI-Tech and clays such as hectorites, for instance, disteardimonium hectorite (and) propylene carbonate sold by the company Elementis under the trade name BENTONE GEL ISD V.

The at least one mineral filler may, in various exemplary embodiments, be present in the cosmetic compositions in an amount ranging from about 0.5% to about 8% by weight, for example, from about 1% to about 6%, or from about 2% to about 5%, or from about 3% to about 5% by weight, relative to the total weight of the composition, including all ranges and subranges therebetween. In various embodiments, the at least one mineral filler is chosen from silica powders and is present in an amount ranging from about 4% to about 5% by weight relative to the total weight of the cosmetic composition.

Cosmetically Acceptable Medium

The cosmetic compositions according to the present disclosure comprise a cosmetically acceptable medium, such as an aqueous or aqueous-alcoholic medium. The cosmetically acceptable medium may, in certain embodiments, comprise water. The amount of water may be present in an amount ranging from about 15% to about 50% by weight, for example, from about 20% to about 40% by weight, or from about 25% to about 35% by weight, relative to the total weight of the composition, including all ranges and subranges therebetween.

The aqueous medium may further comprise at least one organic solvent. In certain embodiments, the organic solvent is water-miscible. Non-limiting examples of suitable organic solvents include C₁-C₄ alkanols, such as ethanol and isopropanol; glycerol; glycols and glycol ethers such as 2-butoxyethanol, propylene glycol, monomethyl ether of propylene glycol, monoethyl ether and monomethyl ether of diethylene glycol; and aromatic alcohols such as benzyl alcohol and phenoxyethanol; analogous products; and mixtures thereof. The organic solvents may be present in an amount ranging from about 1% to about 40% by weight, for example, from about 1% to about 30% by weight, or from about 5% to about 20% by weight, relative to the total weight of the composition, including all ranges and subranges therebetween.

Volatile Solvents

The cosmetic compositions disclosed herein may also comprise at least one volatile solvent. As used herein, the term “volatile solvent” is intended to denote any non-aqueous solvent capable of evaporating on contact with the skin or a keratinous substrate in less than one hour, at approximately room temperature and atmospheric pressure. The at least one volatile solvent disclosed herein are liquid at room temperature, with a non-zero vapor pressure at room temperature and atmospheric pressure, ranging, for example, from 0.13 Pa to 40,000 Pa (10⁻³ to 300 mmHg), such as from 1.3 Pa to 13,000 Pa (0.01 to 100 mmHg), or from 1.3 Pa to 1,300 Pa (0.01 to 10 mmHg). The volatile oils may be chosen, for example, from hydrocarbon-based oils, silicone oils, fluoro oils, and mixtures thereof.

As used herein, the term “hydrocarbon-based oil” means an oil mainly comprising hydrogen and carbon atoms and optionally oxygen, nitrogen, sulfur and/or phosphorus atoms. The volatile hydrocarbon-based oils may be chosen, for example, from hydrocarbon-based oils comprising from 8 to 16 carbon atoms, such as branched C₈-C₁₆ alkanes, for instance C₈-C₁₆ isoalkanes of petroleum origin (also known as isoparaffins), such as isododecane (also known as 2,2,4,4,6-pentamethylheptane), isoeicosane, isooctane, isodecane, and isohexadecane, for example, the oils sold under the trade names Isopar or Permethyl, branched C₈-C₁₆ esters and isohexyl neopentanoate, and mixtures thereof. Other volatile hydrocarbon-based oils, for instance petroleum distillates, such as those sold under the name Shell Solt by the company Shell, may also be used. The volatile solvent may be chosen, in various embodiments, from volatile hydrocarbon-based oils comprising from 8 to 16 carbon atoms, and mixtures thereof.

The volatile oils that may also be used include, for example, volatile silicones, for instance volatile linear or cyclic silicone oils, such as those with a viscosity ≦8 centistokes (8×10⁻⁵ m²/s) and, for example, comprising from 2 to 7 silicon atoms, these silicones optionally comprising at least one group chosen from alkyl and alkoxy groups comprising from 1 to 10 carbon atoms. Among the volatile silicone oils that may be used herein, mention may be made, for example, of octamethyl cyclotetrasiloxane, decamethyl cyclopentasiloxane, dodecamethyl cyclohexasiloxane, hexadecamethyl cyclohexasiloxane, heptamethyl hexyltrisiloxane, heptamethyl octyltrisiloxane, hexamethyl disiloxane, octamethyl trisiloxane, decamethyl tetrasiloxane, dodecamethyl pentasiloxane, and mixtures thereof.

Volatile fluorinated solvents such as nonafluoromethoxybutane or perfluoromethylcyclopentane may, for example, also be used.

The volatile oil may be present in the cosmetic compositions in an amount ranging, for example, from about 1% to about 20% by weight, such as from about 2% to about 15% by weight, for example, from about 6% to about 14% by weight, relative to the total weight of the composition, including all ranges and subranges therebetween. In various embodiments, the at least one volatile solvent is isododecane and is present in an amount ranging from about 2% to about 14% by weight, such as from about 8% to about 14%, or from about 12% to about 14% by weight, relative to the total weight of the cosmetic composition.

Non-Volatile Solvents

The cosmetic compositions disclosed herein may also comprise at least one non-volatile solvent. As used herein, the term “non-volatile solvent” is intended to denote a solvent that remains on the skin or the keratinous substrate at room temperature and atmospheric pressure for at least several hours and that has, for example, a vapor pressure of less than 10⁻³ mmHg (0.13 Pa). In certain embodiments, the non-volatile solvent may be chosen from hydrocarbon-based oils, silicone oils, fluoro oils, and mixtures thereof.

Non-volatile hydrocarbon-based oils that may be mentioned include, for example:

hydrocarbon-based oils of plant origin, such as triglycerides comprising fatty acid esters of glycerol, the fatty acids of which may have varied chain lengths from C₄-C₂₄, these chains possibly being linear or branched, and saturated or unsaturated; these oils are chosen, for example, from wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel,

synthetic ethers comprising from 10 to 40 carbon atoms;

linear or branched hydrocarbons of mineral or synthetic origin, such as petroleum jelly, polydecenes, hydrogenated polyisobutene such as parleam, squalane, and mixtures thereof;

synthetic esters such as oils of formula R₁COOR₂ wherein R₁ is chosen from linear and branched fatty acid residues comprising from 1 to 40 carbon atoms and R₂ is chosen from branched hydrocarbon-based chains comprising from 1 to 40 carbon atoms, provided that the number of carbon atoms is R₁+R₂≧10, such as, purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;

fatty alcohols that are liquid at room temperature, comprising a branched and/or unsaturated carbon-based chain comprising from 12 to 26 carbon atoms, for instance octyldodecanol, isostearyl alcohol, oleyl alcohol, 2-hexyldecanol, 2-butyloctanol and 2-undecylpentadecanol;

higher fatty acids such as oleic acid, linoleic acid and linolenic acid; and

mixtures thereof.

The non-volatile silicone oils that may be used include, for example, non-volatile polydimethylsiloxanes (PDMSs), polydimethylsiloxanes comprising alkyl or alkoxy groups, that are pendent and/or at the end of a silicone chain, wherein the alkyl or alkoxy groups each comprise from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

Non-limiting examples of non-volatile fluoro oils that may be used herein include fluorosilicone oils, fluoropolyethers, and fluorosilicones, for example, those described in European Patent Application Publication No. 0 847 752.

The at least one non-volatile solvent may be present in the cosmetic compositions in an amount ranging from about 1% to about 10% by weight, relative to the total weight of the cosmetic composition. For instance, the at least one non-volatile solvent may be present in an amount ranging from about 2% to about 8% by weight, such as from about 3% to about 6% by weight, relative to the total weight of the cosmetic composition, including any ranges and subranges therebetween. In at least one embodiment, the at least one non-volatile solvent is isononyl isononanoate and is present in the composition in an amount ranging from about 2% to about 4% by weight, for example, from about 2.5% to about 3.5% by weight, relative to the total weight of the cosmetic composition.

Coloring Agents

The cosmetic compositions disclosed herein may further comprise at least one coloring agent. Coloring agents that are suitable in accordance with various exemplary embodiments of the disclosure can produce, for instance, alone or as a mixture, a coloration having a yellow or orange hue. The coloring agents may, in certain embodiments, exhibit a significant reflectance in the range from about 550 to about 675 nm.

The at least one coloring agent may, for example, be chosen from mineral or organic pigments, coloring polymers, water-soluble or liposoluble dyes, organic lacquers, metallic powders, and mixtures thereof. A non-exhaustive list of suitable coloring agents can be found in the CTFA Cosmetic Ingredient Handbook, 3rd Edition Cosmetic and Fragrance Association., Inc., Washington D.C. (1982).

Non-limiting examples of mineral coloring agents include yellow, red and brown metal oxides, for instance, iron oxides and titanium oxides. Metallic powders may include, for example, copper powders. The pigments FDC Yellow No. 5 (disodium salt of tartrazine) may be suitable as organic pigments. Examples of organic lacquers that are suitable for the invention include, for instance, FDC Yellow No. 5 and No. 6 A1 Lake.

The water-soluble dyes may be chosen, for example, from the brown dye identified by the name “caramel” according to the Color Index; the yellow dyes identified by the Color Index numbers 10316, 13015, 18690, 18820, 18965, 19140, 45430, 47005, 75100 and that known as Lactoflavin; the orange dyes identified by the Color Index numbers 14270, 15510, 15980, 15985, 16230, 20170, 40215; the red dyes identified by the Color Index numbers 14700, 14720, 14815, 15620, 16035, 16185, 16255, 16290, 17200, 18050, 18130, 18736, 24790, 27290, 45100, 45220, 45380, 45405, 45410, 45425, 45430, 75470, and mixtures thereof.

The liposoluble dyes may be chosen, for example, from the brown dye identified by the Color Index number 12010; the yellow dyes identified, respectively, by the Color Index numbers 12700, 21230, 47000, 75125, 75135; the orange dyes identified by the Color Index numbers 11920, 40800, 40820, 40825, 40850, 45396, 75120, 75130 and capasanthine and the red dye identified by number 12150, and mixtures thereof.

A dyeing polymer is a polymer comprising at least one organic dye group. The dye group may be grafted, such as via covalent bonding, onto the chain of the polymer. The dye polymer may, in certain embodiments, contain less than about 10% by weight of dyestuff relative to the total weight of the polymer. The dye polymer may be of any chemical nature, such as polyester, polyamide, polyurethane, polyacrylic, poly(meth)acrylic, polycarbonate, of natural origin, for instance, cellulose or chitosan polymers, and mixtures thereof. In certain embodiments, the dye polymer may be a copolymer based on at least two different monomers, at least one of which is an organic dye monomer. Such dye polymers are described, for example, U.S. Pat. Nos. 5,032,670, 4,999,418, 5,106,942, 5,030,708, 5,102,980, 5,043,376, 5,104,913, 5,281,659, 5,194,463, and 4,804,719; International Patent Application Publication No. WO 92/07913; and European Patent Application Publication No. 0 747 036.

Non-limiting examples of dye polymer monomers include anthraquinones, methines, bis-methines, azamethines, arylidenes, 3H-dibenzo[7,i-j]isoquinolines, 2,5-diarylaminoterephthalic acids and esters thereof, phthaloylphenothiazines, phthaloylphenoxazines, phthaloylacridone, anthrapyrimidines, anthrapyrazoles, phthalocyanins, quinophthalones, indophenols, perinones, nitroarylamines, benzodifuran, 2H-1-benzopyran-2-one, quinophthalones, perylenes, quinacridones, triphenodioxazines, fluoridines, 4-amino-1,8-naphthalimides, thioxanthrones, benzanthrones, indanthrones, indigo, thioindigo, xanthene, acridine, azine, and oxazine.

The at least one coloring agent used in the compositions of the instant disclosure may be used in their raw form or may be pretreated, for example by way of surface-treatment. The aim of this treatment is generally to increase the stability of the color and to facilitate their incorporation into cosmetic formulations. In particular, coloring agents treated in order to make them hydrophobic will be more readily dispersible in an oily phase.

For example, the coloring agent may be surface-treated with a hydrophobic and oil-repellant agent of the perfluoroalkyl phosphate derivative type, as described in EP 1 086 683. The coloring agents may also be surface treated with a material that makes them compatible with the oily phases, such as the silicone phases used in certain cosmetic formulations. Pigments of this type are described, for example, in patent U.S. Pat. No. 5,143,722.

Coloring agents suitable for use in accordance with the instant disclosure may include, but are not limited to, brown iron oxide and yellow iron oxide, optionally coated with perfluoroalkyl phosphate, and titanium oxide optionally treated with alumina and/or coated with perfluoroalkyl phosphate, such as the pigmentary pastes sold under the trade names Yellow Iron Oxide Covafluor, PF5 Yellow 601 (yellow) and PF5 Red R516L (red), PF 5 Black BL100 by the company Daito, and those sold under the trade names FA50DRF, FA50DYF, FA65DF and FA65 DBF by the company Kobo; ultramarine blue optionally coated with perfluoroalkyl phosphate, such as the product sold under the trade name PF 5 Ultramarine No. 801 by the company Daito; the disodium salts of tartrazine and the aluminium lakes of Allura red on alumina sold by the company Noveon under the names FDC Yellow No. 6, A1 Lake and FDC Yellow No. 5 A1 Lake, and mixtures thereof.

Alternatively the coloring agent may be chosen from those treated with at least one amino acid, which may exhibit improved affinity for the skin for example, pigments from Miyoshi Kasei sold under the names NA1-C33-8001-10, NAI-C33-8001-10, NA1-C33-7001-10, and NA1-C33-9001-10.

It is within the ability of one skilled in the art to select the coloring agents and their amounts to adjust the color of the composition to create the desired effect, for example to adjust the bulk color of the composition and/or the color of the composition on the skin. Generally, the at least one coloring agent may be present in the composition in an amount ranging from about 0.5% to about 30% by weight, for example, from about 2% to about 20% by weight, or from about 5% to about 15% by weight.

One embodiment of the disclosure relates to cosmetic compositions comprising (1) at least one silicone elastomer blend chosen from dimethicone cross-polymers dispersed in a silicone oil, (2) at least one film-forming polymer chosen from polypropylsilsesquioxane resins, (3) at least one siloxane resin chosen from vinyl dimethicone/methicone silsesquioxane cross-polymers, (4) at least one polymeric filler chosen from polyamide powders, and (5) at least one mineral filler chosen from silica powders.

In one exemplary embodiment, the silicone elastomer blend may, by way of example, be a dimethicone cross-polymer dispersed in dimethicone, such as, for example, Dow Corning® 9041 sold by Dow Corning. In various exemplary embodiments, the at least one polypropylsilsesquioxane resin may be Dow Corning® 680 ID Fluid sold by Dow Corning. In other exemplary embodiments, the at least one siloxane resin may be chosen from products sold by Shin-Etsu under the KSP product line, for example, KSP-100, KSP-101, and KSP-105. In yet another exemplary embodiment, the at least one polymeric filler may, by way of example, be a Nylon powder, such as, for example Nylon-12 from Arkema. In a further embodiment, the at least one mineral filler may be chosen from silica microspheres, such as those sold by AGC SI-Tech under the name Sunsphere® H51.

In addition, other cosmetic ingredients may be included in the compositions according to the disclosure. Such ingredients are known, and include but are not limited to humectants, emulsifiers, surfactants, preservatives, fragrances, thickeners or texturizers, fatty compounds, emollients, coalescents, and/or plasticizers. One of skill in the art will be able to select appropriate types and amounts of additional cosmetic ingredients, based on, for example, the type of cosmetic composition being formulated and the desired properties thereof. By way of example only, such additional cosmetic ingredients may be present in the compositions according to the disclosure in a combined amount ranging from about 10% to about 80%, such as about 15% to about 60%, about 25% to about 40%, or about 30% to about 35%, including all ranges and subranges therebetween.

Exemplary cosmetic compositions contemplated according to the disclosure include compositions are intended for application to a keratinous substrate, such as the skin. Such compositions may include, but are not limited to, make-up compositions, such as liquid foundations and powders.

Without wishing to be bound by theory, it is believed that the combination of the at least one polyalkylsilsesquioxane resin, at least one siloxane resin, and at least one silicone elastomer blend as described herein surprisingly and unexpectedly allows for the incorporation of polymeric and/or mineral fillers in amounts sufficient to absorb oil and/or reduce shine while also reducing the appearance of ashiness and/or dullness of the skin. By way of example only, foundation formulations described herein have been found to have one or more improved properties, such as improved oil absorption, shine reduction, matte, texture, long-wear properties, and/or ability to impart true color, while also reducing dullness, whiteness, and/or ashiness of the skin. It should be noted, however, that compositions according to the disclosure may not have one or more of the above-referenced improved properties, yet such compositions are intended to be within the scope of the disclosure.

Cosmetic Method

The instant disclosure also relates to a cosmetic method for making up and/or enhancing the appearance of a keratinous substrate, the method comprising applying to the keratinous substrate a composition comprising, in a cosmetically acceptable medium, (1) at least one silicone elastomer blend, (2) at least one film-forming polymer chosen from polyalkylsilsesquioxane resins, (3) at least one siloxane resin, (4) at least one polymeric filler, and (5) at least one mineral filler. All embodiments disclosed above with respect to the cosmetic compositions are equally applicable to the cosmetic method and are intended to fall within the scope of the disclosure.

According to one embodiment of the present disclosure, a cosmetic method for making up the skin, such as the facial skin, is provided. This method may, in certain embodiments, comprise the steps of loading an applicator, such as a sponge or pad, with a cosmetic composition disclosed herein, and applying said cosmetic composition onto the skin. In other embodiments, the user may apply the composition to the skin using the fingers. According to certain embodiments, the cosmetic composition may be applied to the skin more than once, such as twice or more, according to the cosmetic effect desired by the user.

The cosmetic composition according to the invention may be packed in a cosmetic container delimiting at least one compartment which comprises the cosmetic composition, the container being closed by a closing member.

It is to be understood that both the foregoing description and the following Examples are exemplary and explanatory only, and are not to be interpreted as restrictive of the disclosure. Moreover, it should be understood that various features and/or characteristics of differing embodiments herein may be combined with one another. It is therefore to be understood that numerous modifications may be made to the illustrative embodiments and that other arrangements may be devised without departing from the scope of the invention. Other embodiments will be apparent to those skilled in the art from consideration of the disclosure and practice of the various exemplary embodiments disclosed herein.

It is also to be understood that, as used herein the terms “the,” “a,” or “an,” mean “at least one,” and should not be limited to “only one” unless explicitly indicated to the contrary. Thus, for example, the use of “a siloxane resin” is intended to mean at least one siloxane resin.

Unless otherwise indicated, all numbers used in the specification and claims are to be understood as being modified in all instances by the term “about,” whether or not so stated. It should also be understood that the precise numerical values used in the specification and claims form additional embodiments of the invention, and are intended to include any ranges which can be narrowed to any two end points disclosed within the exemplary ranges and values provided. Efforts have been made to ensure the accuracy of the numerical values disclosed herein. Any measured numerical value, however, can inherently contain certain errors resulting from the standard deviation found in its respective measuring technique.

### Examples

The following Examples are intended to be non-restrictive and explanatory only, with the scope of the invention being defined by the claims.

Liquid Foundation Composition

The following composition was prepared by mixing, independently, the components set forth in the following Table 1.

TABLE 1
INVENTIVE COMPOSITION

| Component | Wt % |
|---|---|
| Pigments | 12 |
| Sodium Chloride | 0.7 |
| Dicaprylyl Ether | 4.35 |
| Mineral Fillers | 10.0 |
| Polymeric Filler | 2.0 |
| Siloxane Resin | 1.0 |
| Silicone | 3.0 |
| Silicone Elastomer Blend | 13.0 |
| Polypropylsilsesquioxane (72% A.M.) | 4.5 |
| Volatile Solvent | 13.43 |
| Non-volatile Solvents | 4.0 |
| Water | 25.02 |
| Alcohol Denat. | 7 |

Instrumental Evaluation

Method

The inventive composition and a comparative composition (True Match by L′Oreal) were subjected to instrumental testing. True Match is a similar foundation composition, but does not comprise a siloxane resin or a silicone elastomer blend as described herein.

Each composition was applied to one-half of the face of 16 women with oily skin, ages 18-60. Each composition was applied in an amount of 0.70 mg/cm³, or approximately 100 mg per half of the face. The matte appearance of the skin was evaluated at three separate times, T₀ (baseline, bare skin 15 minutes after cleansing),

Tᵢₘₘ (made-up skin 10 minutes after makeup application), and T₃ₕᵣ (made-up skin 3 hours after makeup application), using a chromasphere and polarimetric camera.

Results

The difference of matte appearance between time points was determined for Tᵢₘₘ-T₀, T₃ₕᵣ-T₀, and T₃ₕᵣ-Tᵢₘₘ and is expressed as an average in Table 2 below. Negative values indicate that the skin had a higher matte appearance in the later measurement. For example, in the case of Tᵢₘₘ-T₀, a negative value indicates that the skin was more matte at Tᵢₘₘ than at T₀. Similarly, positive values indicate that the skin had a higher matte appearance in the earlier measurement. For instance, in the case of T₃ₕᵣ-Tᵢₘₘ, a positive value indicates that the skin was more matte at Tᵢₘₘ than at T₃ₕᵣ.

TABLE 2
MATTE APPEARANCE - INSTRUMENTAL (TRUE MATCH)

| | | | Matte |
|---|---|---|---|
| | Matte Appearance | Matte Appearance | Appearance |
| | Tᵢₘₘ-T₀ | T_{3 hr}-T₀ | T_{3 hr}-Tᵢₘₘ |
| L'Oreal True Match | −3.66 ± 1.70 | 0.10 ± 2.65 | 3.76 ± 2.22 |
| Inventive | −5.84 ± 2.46 | −2.56 ± 3.91 | 3.28 ± 2.40 |
| Composition | | | |

As shown in Table 2 above, when evaluating the immediate effect of the compositions on the matte appearance of the skin (Tᵢₘₘ-T₀), the inventive composition substantially outperformed L'Oréal True Match. Similarly, when evaluating the long wear matte effect of the compositions (T₃ₕᵣ-T₀), the inventive composition provided a substantially greater matte effect as compared to L'Oréal True Match. These values were analyzed for statistical significance using the Student's t-test (a=0.05) and determined to be statistically different. Finally, when evaluating the matte appearance with wear over time (T₃ₕᵣ-Tᵢₘₘ), it was determined that the inventive composition and True Match performed similarly, i.e., the values were not significantly different from each other, as analyzed using the Student's t-test (a=0.05).

Accordingly, the inventive composition was found to be unexpectedly superior to L'Oréal's True Match product, in terms of providing an improved matte appearance to the skin both upon immediate application of the composition and after a period of long wear, as evaluated by instrumental analysis.

Consumer Panel Monadic Evaluation

Method

Three different shades of the inventive composition were prepared by varying the amount and type of pigments used in the formulations, but without substantially changing the overall formulation presented in Table 1 above. The three shades (light, medium, and dark) were tested by a panel of 34 women of color, according to the shade of their skin tone. 3 women evaluated the light shade, 25 women evaluated the medium shade, and 6 women evaluated the dark shade.

Results

The panel was asked to evaluate the inventive composition on its own, without comparison to any other product. The results of this evaluation are illustrated in the following Tables 4 and 5. In Table 4, qualitative measures of various cosmetic properties were provided by the consumers. In Table 5, quantitative measures of various cosmetic properties were provided by the consumers, on a scale of 1 to 9, with 1 being very dissatisfied and 9 being very satisfied. The mean value among the 34 women for each property was calculated and is represented below in Table 5.

TABLE 4
CONSUMER EVALUATION - QUALITATIVE ANALYSIS

| PROPERTY | FREQUENCY | PERCENTAGE |
|---|---|---|
| Ease of Application | | |
| Very easy to apply | 28 | 82% |
| Somewhat easy to apply | 6 | 18% |
| Neither difficult nor easy to apply | 0 | 0% |
| Somewhat difficult to apply | 0 | 0% |
| Very difficult to apply | 0 | 0% |
| Evenness of Application | | |
| Very even | 24 | 71% |
| Somewhat even | 8 | 24% |
| Somewhat uneven | 1 | 3% |
| Very uneven | 1 | 3% |
| Moist Feel | | |
| Very moisturized | 10 | 29% |
| Somewhat moisturized | 17 | 50% |
| Neither moisturized nor dry | 5 | 15% |
| Somewhat dry | 2 | 6% |
| Very dry | 0 | 0% |

TABLE 5
CONSUMER EVALUATION - QUANTITATIVE ANALYSIS

| PROPERTY | MEAN RATING |
|---|---|
| Satisfaction with application | 7.559 |
| Satisfaction with overall texture | 8.029 |
| Silkiness of texture | 7.735 |
| Smoothness of texture | 8.059 |
| No oily/greasy feel | 8.500 |
| Soft/smooth/silky feel | 7.882 |
| No streaking | 8.235 |
| No ashiness | 7.765 |
| Natural appearance | 7.235 |

As shown in Tables 4 and 5 above, a large majority of users found the product to be very satisfactory, providing a non-ashy, natural appearance to the skin, while also having a pleasing texture, non-greasy feel, and moisturizing effect. Accordingly, the inventive composition surprisingly provides a matte texture to skin of deeper ethnic tones, while also providing oil and/or shine control, and reducing the appearance of ashiness of the skin.

Expert Panel Comparative Evaluation

Method

A panel of ten sensory experts evaluated the cosmetic properties of the inventive composition as compared to True Match by L'Oréal, including application, skin feel, appearance, and wear attributes. The panelists were experts trained in the application and evaluation of liquid foundation products, able to detect small difference not always perceivable by the average consumer. The compositions were evaluated using a sequential monadic test design in which the samples were presented to panelists in randomized order within the replication block and across panelists.

The panelists first used a standard cleanser to wash their faces before product application. The skin was allowed to equilibrate for 10 minutes. The panelists applied 0.025 ml of product to half of the forehead, 0.050 ml of product to one cheek, and 0.025 ml of the product to one half of the jaw line using their fingers. The product was then smoothed or blended as appropriate for the evaluation.

The compositions were rated using a 15-point universal intensity scale, where 0 represents no intensity and 15 represents high intensity. The compositions were evaluated during and after application, as well as after 4 and 8 hours of wear. Each evaluation was performed in duplicate and a mean value calculated. The results of this testing are illustrated in the following Table 6. Statistically significant differences (95% confidence; p-value=0.05) are bolded and shaded in gray.

TABLE 6
EXPERT EVALUATION—COMPARISON TO TRUE MATCH

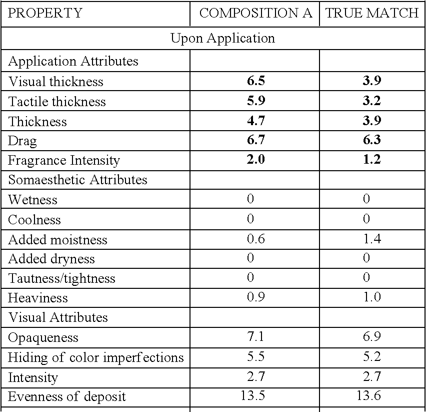

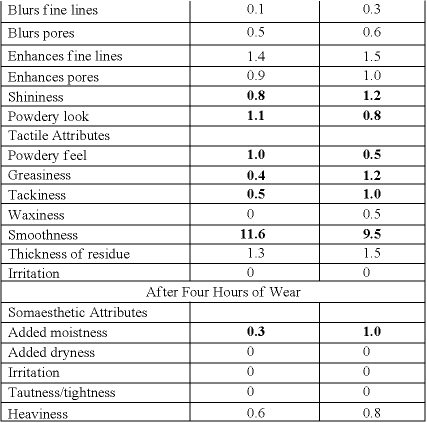

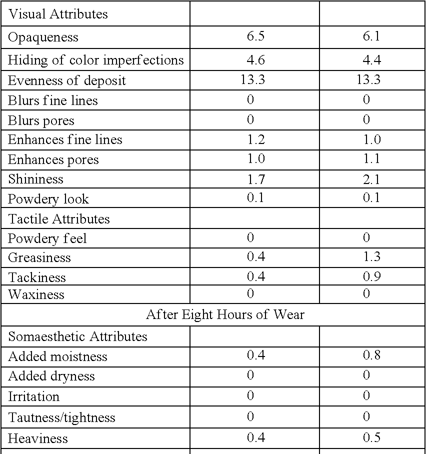

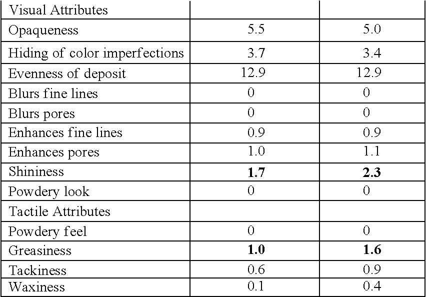

As shown in Table 6 above, according to the expert panel, True Match by L'Oréal produced a cosmetic effect that was significantly more greasy and shiny at all points of evaluation (upon application, after 4 hours of wear, and after 8 hours of wear) as compared to the inventive composition. In addition, L'Oréal True Match was found to impart significantly more tackiness and added moisture upon application and after 4 hours of wear. Accordingly, the inventive composition was surprisingly found to have improved oil and/or shine reduction as compared to the commercial product True Match by L'Oréal (not comprising a siloxane resin or silicone elastomer blend as described herein).

1. A cosmetic composition comprising, in a cosmetically acceptable medium:
(1) at least one silicone elastomer blend, present in an amount ranging from about 5% to about 15% by weight, relative to the total weight of the cosmetic composition,
(2) at least one film-forming polymer chosen from polyalkylsilsesquioxane resins, present in an amount ranging from about 2% to about 10% by weight, relative to the total weight of the cosmetic composition,
(3) at least one siloxane resin chosen from vinyl dimethicone/methicone silsesquioxane cross-polymers, present in an amount ranging from about 0.5% to about 3% by weight, relative to the total weight of the cosmetic composition,
(4) at least one polymeric filler, present in an amount ranging from about 0.5% to about 5% by weight, relative to the total weight of the cosmetic composition, and
(5) at least one mineral filler, present in an amount ranging from about 0.5% to about 8% by weight, relative to the total weight of the cosmetic composition.

2. The cosmetic composition of claim 1, wherein the at least one silicone elastomer blend is chosen from silicone cross-polymers dispersed in silicone oils.

3. The cosmetic composition of claim 2, wherein the at least one silicone elastomer blend is a dimethicone cross-polymer dispersed in dimethicone.

4. (canceled)

5. The cosmetic composition of claim 1, wherein the at least one polyalkylsilsesquioxane resin is chosen from polypropylsilsesquioxane resins optionally dispersed in a carrier solvent chosen from isoparaffins, isododecane, isoeicosane, and isohexadecane.

6. (canceled)

7. The cosmetic composition of claim 1, further comprising at least one additional film-forming polymer chosen from acrylic polymers.

8. (canceled)

9. (canceled)

10. The cosmetic composition of claim 1, wherein the at least one polymeric filler is chosen from polyamide powders.

11. (canceled)

12. The cosmetic composition of claim 1, wherein the at least one mineral filler is chosen from silica powders.

13. (canceled)

14. The cosmetic composition of claim 1, further comprising (a) at least one volatile solvent chosen from volatile hydrocarbon-based oils, silicone oils, fluoro oils, and mixtures thereof and/or (b) at least one non-volatile solvent chosen from non-volatile hydrocarbon-based oils, silicone oils, fluoro oils, and mixtures thereof.

15. The cosmetic composition of claim 14, wherein (a) the at least one volatile solvent, when present, is present in the composition in an amount ranging from about 1% to about 20% by weight, relative to the total weight of the composition, and (b) the at least one non-volatile solvent, when present, is present in the composition in an amount ranging from about 1% to about 10% by weight, relative to the total weight of the composition.

16. The cosmetic composition of claim 1, further comprising at least one coloring agent chosen from mineral or organic pigments, coloring polymers, water-soluble or liposoluble dyes, organic lacquers, metallic powders, and mixtures thereof.

17. The cosmetic composition of claim 16, wherein the at least one coloring pigment is present in the composition in an amount ranging from about 0.5% to about 30% by weight, relative to the total weight of the composition.

18. A cosmetic composition comprising, in a cosmetically acceptable medium:
(1) at least one silicone elastomer blend chosen from dimethicone cross-polymers dispersed in dimethicone, present in an amount ranging from about 5% to about 15% by weight, relative to the total weight of the cosmetic composition,
(2) at least one polypropylsilsesquioxane film-forming polymer, present in an amount ranging from about 2% to about 10% by weight, relative to the total weight of the cosmetic composition,
(3) at least one vinyl dimethicone/methicone silsesquioxane cross-polymer, present in an amount ranging from about 0.5% to about 3% by weight, relative to the total weight of the cosmetic composition,
(4) at least one polyamide powder, present in an amount ranging from about 0.5% to about 5% by weight, relative to the total weight of the cosmetic composition, and
(5) at least one silica powder, present in an amount ranging from about 0.5% to about 8% by weight, relative to the total weight of the cosmetic composition.

19. A method for making up and/or enhancing the appearance of a keratinous substrate comprising applying to the keratinous substrate a cosmetic composition comprising, in a cosmetically acceptable medium:
(1) at least one silicone elastomer blend, present in an amount ranging from about 5% to about 15% by weight, relative to the total weight of the cosmetic composition,
(2) at least one film-forming polymer chosen from polyalkylsilsesquioxane resins, present in an amount ranging from about 2% to about 10% by weight, relative to the total weight of the cosmetic composition,
(3) at least one siloxane resin chosen from vinyl dimethicone/methicone silsesquioxane cross-polymers, present in an amount ranging from about 0.5% to about 3% by weight, relative to the total weight of the cosmetic composition,
(4) at least one polymeric filler, present in an amount ranging from about 0.5% to about 5% by weight, relative to the total weight of the cosmetic composition, and
(5) at least one mineral filler, present in an amount ranging from about 0.5% to about 8% by weight, relative to the total weight of the cosmetic composition.

20. The method of claim 19, wherein the cosmetic composition is a foundation and the keratinous substrate is the skin.
